(19) United States
(12) Patent Application Publication
(10) Pub. No.: US 2007/0265498 A1
(43) Pub. Date: Nov. 15, 2007

(54) ELECTRONIC ENDOSCOPE CONNECTING MECHANISM

(75) Inventor: Shunichi ITO, Tokyo (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/746,138

(22) Filed: May 9, 2007

(30) Foreign Application Priority Data
May 10, 2006 (JP) 2006-131594

(51) Int. Cl. A61B 1/00; A61B 1/04; H04N 7/18

(52) U.S. Cl. 600/134; 600/132; 348/E05.044; 348/75

(57) ABSTRACT

A connecting mechanism for an electronic endoscope system is provided. The connecting mechanism is for detachably connecting a scope and a processor unit of the electronic endoscope system. The connecting mechanism includes a rotational cylinder, a linear movable member, and a sensor. The rotational cylinder locks the scope-side connector section to the processor-side connector section by rotating the rotational cylinder, when the scope-side connector section is docked with the processor-side connector section. The linear movable member is moved linearly along a tangent of the rotational cylinder in cooperation with the rotation of the rotational cylinder. The sensor detects whether the linear movable member has arrived at a predetermined position. The predetermined position corresponds to a locking position where the scope-side connector section and the processor-side connector section are locked together by the rotational cylinder.

### BACKGROUND OF THE INVENTION

1. Field of the Invention

The present invention relates to a connector for detachably connecting an endoscope to a processor unit.

2. Description of the Related Art

In an electronic endoscope system, the processor unit, which is used to process image signals from a scope, is generally set up for use as a separate unit, and the scope is set up for use as a device that can be detachably attached to the processor unit. When connecting the scope to the processor unit, the cylindrical connector of the scope is docked with the cylindrical connector of the processor unit, and a lock lever provided on the cylindrical connector of the processor unit is rotated so that the cylindrical connector of the scope securely locks into the cylindrical connector of the processor by means of a bayonet mount mechanism.

Since communication between the processor unit and the scope should only be started after establishing a secured mechanical connection between the connectors, determination of whether or not the connectors are securely docked together is required. Conventionally, this determination is achieved by the signal from a limit switch that is engaged by a protuberance provided on the periphery of the connector provided on the processor unit, and such that the limit switch is operated by the rotation of the cylindrical connector of the processor unit.

### SUMMARY OF THE INVENTION

In the conventional connection structure above, the engagement of the limit switch is effected directly by the arced movement of the connector, which means that the position of the limit switch must be accurately set during assembly of the device. Particularly, a cylindrical connector, subjected to rotational operation, requires a large tolerance and is subjected to play of movement. This results in the connector not being stable enough to enable easy accurate positioning of the limit switch.

Consequently, an object of the present invention is to provide a connecting mechanism for an electronic endoscope that does not require fine position adjustment when installing the sensor whilst still reliably detecting whether or not the connector is locked.

According to the present invention, a connecting mechanism for an electronic endoscope system is provided. The connecting mechanism is for detachably connecting a scope to the processor unit of an electronic endoscope system. The connecting mechanism includes a rotational cylinder, a linear movable member, and a sensor. The rotational cylinder locks the scope-side connector section (the connector provided on the scope) to the processor-side connector section (the connector provided on the processor) by turning the rotational cylinder when the scope-side connector section is fully inserted into the processor-side connector section. The linear movable member is displaced linearly along a tangent of, and in co-operation with, the movement of the rotational cylinder. The sensor detects whether the linear movable member has arrived at a predetermined position, which corresponds to the locking position where the scope-side connector section and the processor-side connector section are locked together by the rotational cylinder.

### BRIEF DESCRIPTION OF THE DRAWINGS

The objects and advantages of the present invention will be better understood from the following description, with reference to the accompanying drawings in which:

FIG. 1 is a front view of the processor-side connector that is provided on the processor unit;

FIG. 2 is a side cross-sectional view of the processor-side connector; and

FIG. 3 is a plan view only illustrating the relationship between the casing, the sliding plate, and the sensor.

### DESCRIPTION OF THE PREFERRED EMBODIMENTS

The present invention is described below with reference to the embodiments shown in the drawings.

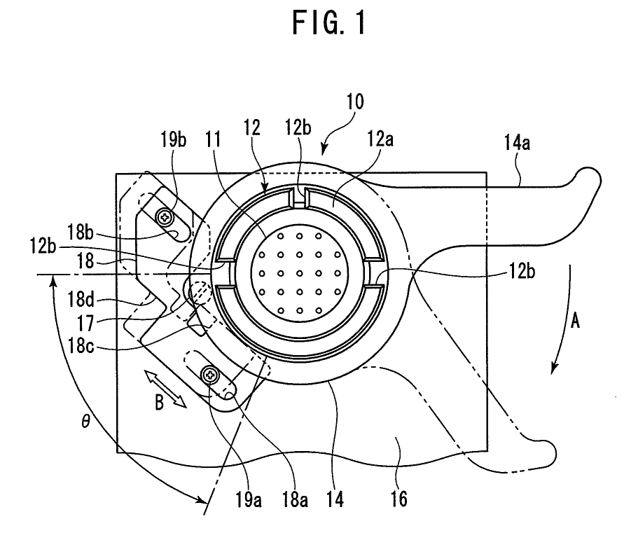

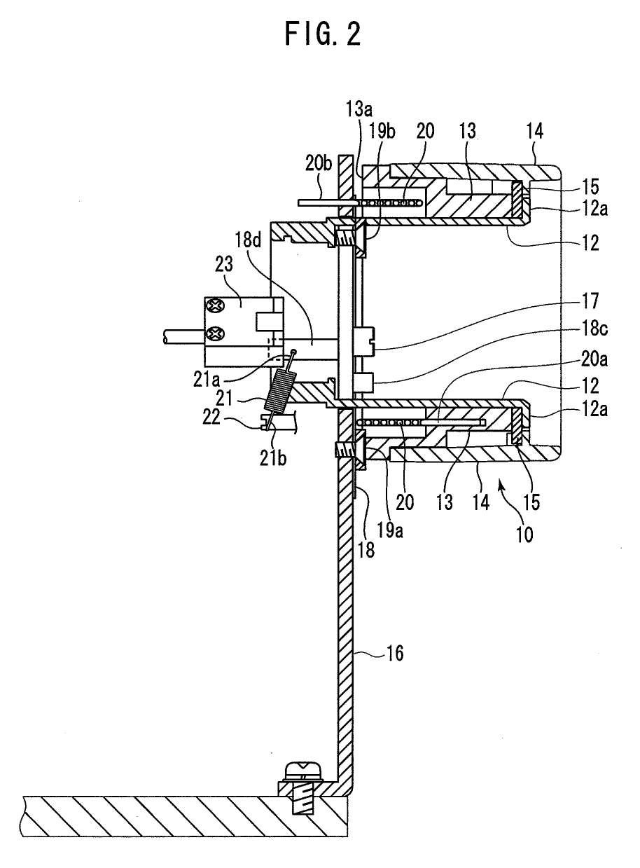

FIG. 1 is the front view of a connector (the processor-side connector) 10 that is provided on the surface of the processor unit of the present embodiment. Further, FIG. 2 is a side cross-sectional view of the connector 10. In FIG. 2, members which do not actually exist in this section are also depicted.

At the center of the processor-side connector 10, there is provided a cylindrical socket 11 (only shown in FIG. 1), where a plurality of terminals (not shown) that are provided on the scope-side connector are plugged into. Namely, a plurality of pinholes are formed in the end face of the socket 11 and each of the terminals is inserted into the pinholes. Around the socket 11, a cylindrical retainer 12 is coaxially located. The front end of the cylindrical retainer 12 is provided with a flange 12a that extends radially outside. The base end of the cylindrical retainer 12 penetrates the casing 16 of the processor unit and the cylindrical retainer 12 is fixed to the casing 16 via a support member (not shown).

A first rotational cylinder 13 is fitted around the periphery of the cylindrical retainer 12 and rotationally supported by the cylindrical retainer 12. Further, a second rotational cylinder 14 that is provided with a lever 14a is fitted around the periphery of the first rotational cylinder 13. The first and second rotational cylinders 13 and 14 are fitted together, so that they are integrally rotated about the cylindrical retainer 12 when the lever 14a is rotated (denoted as “A” in FIG. 1). Note that a sliding member 15 is inserted between the end face of the first rotational cylinder 13 and the flange 12a.

In the present embodiment, three notches 12b are formed on the flange 12a and tabs (not shown) that are provided on the scope-side connector are inserted therethrough. Namely, the scope-side connector is securely attached to the processor-side connector 10 by a bayonet mount mechanism, such that the tabs are engaged with the flange 12a when the lever 14a is rotated in the direction “A” as shown in FIG. 1.

In FIG. 1, the initial position of the lever 14a is indicated by a solid line and the locking position, a position where the connectors are securely fixed together, is indicated by a phantom line. Further, in the locking position, the position of the first and second rotational cylinders 13 and 14 is retained by a plunger mechanism (not shown).

On the casing 16, at a position where the end face 13a of the base end of the first rotational cylinder 13 is located, a pin 17 that protrudes from the casing 16 is provided as a stop member. However, a recessed portion is formed along the arcuate section of the end face 13a so that the pin 17 is prevented from contact with the end face 13a within the range “θ” of FIG. 1. Namely, the range of rotational operation of the lever 14a is restricted by the contact between the pin 17 and the either edges of the recessed portion.

Further, a sliding plate (the linear movable member) 18 formed of a thin plate is laid on the surface of the casing 16. In the present embodiment, the sliding plate 18 is provided with two guide slits 18a and 18b. Pins 19a and 19b, which are screwed on the casing 16, engages with each of the guide slits 18a and 18b so that the sliding plate 18 is retained but linearly movable along the length of the guide slits 18a and 18b (a direction “B” of FIG. 1).

The sliding plate 18 is provided with bent portions 18c and 18d. The bent portion 18c is folded outwards toward the first rotational cylinder 13 and extends inside the recessed portion, which is formed on the end face 13a of the base end of the first rotational cylinder 13. On the other hand, the bent portion 18d is folded inwards toward the casing 16. Further, the plane of the bent portion 18d is arranged parallel with the axes of the guide slits 18a and 18b, and the plane of the bent portion 18c is arranged perpendicular to the axes.

When the lever 14a is rotated in the direction “A” from its initial position and the first rotational cylinder 13 is rotated in the clockwise direction, one edge of the recessed portion on the end face 13 of the base end of the first rotational cylinder 13 engages the bent portion 18c. Thereby, the sliding plate 18 is urged toward the upper-left side along the guide slits 18a and 18b in FIG. 1.

Note that the initial position of the sliding plate 18, i.e., the position of the sliding plate 18 when the lever 14a is at its initial position, is indicated by a solid line. On the other hand, the locking position of the sliding plate 18, i.e., the position of the sliding plate 18 when the lever 14a is rotated to the locking position, is indicated by a phantom line. Further, the direction of linear motion of the sliding plate 18 at the position where an edge of the recessed portion and the bent portion 18c engage, is a tangent of an arc centered on the axis of the first and second rotational cylinders 13 and 14.

Biasing members 20 and 21 are provided respectively on the first rotational cylinder 13 and the sliding plate 18, so that each of the members is biased from the fastening position to the initial position. Namely, the first rotational cylinder 13 is rotationally biased by a torsion spring 20 in the counter direction of the direction “A” (the counter clockwise direction in FIG. 1). Additionally, the sliding plate 18 is biased by a spring 21 in the lower-right direction along the direction “B” in FIG. 1, thereby, the sliding plate 18 abuts the edge of the recessed portion of the end face 13a.

Note that one end 20a of the torsion spring 20 engages the first rotational cylinder and the other end 20b is fixed to the casing 16 via a retainer (not shown). Further, one end 21a of the spring 21 is attached to the bent portion 18d and the other end 21b is attached to a retainer 22 that is fixed to the casing 16.

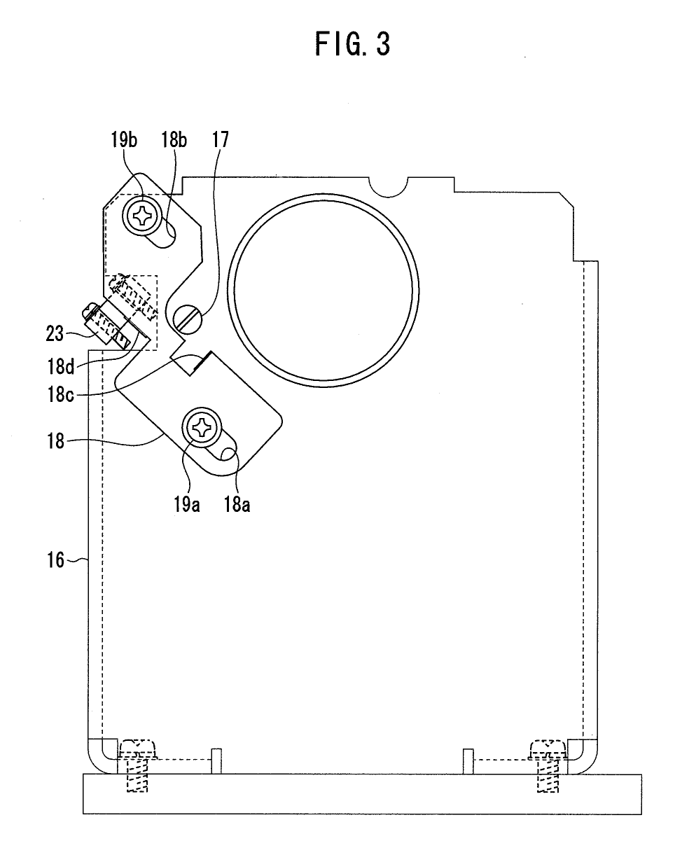

Referring to FIGS. 2 and 3, the structures that are utilized to detect whether the lever 14a has rotated to the locking position will be explained. FIG. 3 is a plan view only illustrating the relationship between the casing 16, the sliding plate 18, and a sensor 23.

As shown in FIGS. 2 and 3, a photointerrupter, which is a non-contact type area sensor, is positioned inside the casing 16 as the sensor 23. The sensor 23 is so arranged to emit light in a direction perpendicular to the linear motion of the sliding plate 18 and the bent portion 18d. The bent portion 18d is caused to pass between the light emitter and the light receiver of the sensor 23 during the linear motion of the sliding plate 18.

Namely, when the sliding plate 18 is at its initial position, the bent portion 18d does not interrupt the light of the sensor 23. Thus, when the lever 14a is rotated and the sliding plate 18 reaches the locking position, the bent portion 18d is positioned between the light emitter and the light receiver of the sensor 23, and interrupts the light. Consequently, the fact that the lever 14a has been rotated to the locking position is detected.

As described above, according to the present embodiment, the point when the lever has reached the locking position is determined by detecting the position of the sliding plate, which is cooperated by the rotation of the lever, so that the condition of whether or not the connectors are locked together is are more reliably detected. Further, the effect of positional variance of the rotational members, which is caused by tolerance bore in the rotational mechanism, is also reduced so that a fine adjustment, which is usually necessary to carry out when assembling the sensor, becomes unnecessary.

Although the embodiment of the present invention has been described herein with reference to the accompanying drawings, obviously many modifications and changes may be made by those skilled in this art without departing from the scope of the invention.

The present disclosure relates to subject matter contained in Japanese Patent Application No. 2006-131594 (filed on May 10, 2006), which is expressly incorporated herein, by reference, in its entirety.

1. A connecting mechanism for an electronic endoscope system that is used for detachably connecting a scope and a processor unit of said electronic endoscope system, said connecting mechanism comprising:
a rotational cylinder that locks a scope-side connector section to a processor-side connector section by turning said rotational cylinder, when the scope-side connector section is docked with said processor-side connector section;
a linear movable member that is moved linearly along a tangent of the arc of movement of said rotational cylinder in cooperation with the rotation of said rotational cylinder; and
a sensor that detects whether said linear movable member has arrived at a predetermined position;
wherein said predetermined position corresponds to a locking position where said scope-side connector section and said processor-side connector section are locked together by said rotational cylinder.

2. The connecting mechanism as claimed in claim 1, wherein said sensor comprises a non-contact type sensor.

3. The connecting mechanism as claimed in claim 1, wherein said rotational cylinder comprises an engaging portion that directly engages said linear movable member, and said linear movable member is biased by a biasing member and abuts against said engaging portion.

4. The connecting mechanism as claimed in claim 1, further comprising a biasing member that rotationally biases said rotational cylinder away from said locking position to an initial position where said scope-side connector section can be docked with said processor-side connector section.
